(19) United States
(12) Patent Application Publication
(10) Pub. No.: US 2001/0005509 A1
(43) Pub. Date: Jun. 28, 2001

(54) Foot cream composition

(76) Inventor: Marie Harbeck, Austin, TX (US)

Correspondence Address: MARIE HARBECK, 3202 CLUMPGRASS COVE, AUSTIN, TX, 78735, US

(21) Appl. No.: 09/753,900

(22) Filed: Jan. 3, 2001

Related U.S. Application Data
(63) Continuation-in-part of application No. 09/248,573, filed on Feb. 11, 1999, now Pat. No. 6,193,987.

(51) Int. Cl. A61K 35/78; A01N 65/00; A61K 47/00; A01N 25/00; C01B 35/10; C07C 35/18; C07C 49/76; C07C 49/84; C07C 49/82; C07C 49/786

(52) U.S. Cl. 424/401; 424/747; 424/735; 514/458; 424/768; 424/770; 424/736; 514/787; 424/727; 424/746; 424/765; 568/824; 423/277; 548/317.1; 568/331

(57) ABSTRACT

An improved, and useful, topical transdermal delivery composition for the alleviation and treatment of dermatitis, eczema, lupus, dry, cracked, flaking, peeling, and itching skin infirmites of the foot, which has as its constituents, safflower oil, flaxseed oil, vitamin A, tocopheryl linoleate, and tincture of benzoin to restore organic oil lipids that are essential for the health of the dermal areas of the foot.

This is a “Continuation-In-Part” application for Ser. No. 09/248,573 filed Feb. 11, 1999 for Marie Harbeck.

### BACKGROUND

1. Field of Invention

This invention comprises an improved and useful topical transdermal delivery system for the alleviation and treatment of dermatitis, eczema, lupus, dry, cracked, flaking, peeling, and itching skin infirmites of the foot, which has as main constituents, safflower oil, flaxseed oil, vitamin A, tocopheryl linoleate, and tincture of benzoin.

To the best of the applicant's knowledge the following is the most relevant prior art:

| | | |
|---|---|---|
| U.S. Pat. No. 4,740,367 | Force, et al. | 1988 |
| U.S. Pat. No. 5,244,679 | Freston. | 1993 |
| U.S. Pat. No. 5,489,429 | Griat, et al. | 1996 |
| U.S. Pat. No. 5,683,704 | Ohba, et al. | 1997 |
| U.S. Pat. No. 4,883,659 | Goodman, et al. | 1989 |
| U.S. Pat. No. 4,608,392 | Jacquet et al. | 1986 |
| U.S. Pat. No. 5,431,911 | Reynolds. | 1995 |

The cosmetic and pharmaceutical industry is continually expanding its efforts in order to provide topical preparations which will moisten and soften the skin, eliminate dryness, cracking, chapping, chaffing, redness and restore the skin to its natural healthy condition.

Thus, synthetic surfactants, silicones, ammonia, alcohol's, solvents, acids, artificial colors and fragrances, and the like are being incorporated into the compositions of the cited prior art that are supplied to stores and supermarkets.

Some of these components agrravate skin infirmities thus the combinations of skin aggravating synthetic compositions employed continue to increase yearly.

It will be appreciated that the expansion and growth of the cited prior art has devolved into highly complex synthetic adjuvants for use in the formulations.

The problem with the use of the compositions of the cited prior art which make up the bulk of transdermal delivery systems are their chemical structures which are known to cause foot, and skin irritations.

Alcohol, acids, solvents, synthetic perfumes and colorants, and ammonia adjuvants incorporated into the compositions of the prior art cause dryness of the skin, rashes, allergies, eczema, dermatitis, peeling, bleeding, itching, painful cracking and flaking of the skin.

Unfortunately, the cited prior art compositions are relatively ineffective for distressed skin conditions, not allowing the treatment and alleviation of skin irritations related to allergic skin reactions to synthetic adjuvants, so there is a need in the art for organic compounds for improved topical treatments for foot care.

The said prior art compositions do not employ massive quantities of cold-pressed organic vegetable oils as in the present invention.

The said prior art compositions do not employ organic beeswax compositions that this invention incorporates due to prohibitive manufacturing costs.

In terms of effectiveness, the said cited prior art compositions do not provide satisfactory skin penetration and supplying properties to leave the feet satiny smooth for up to twelve hours.

The said cited prior art compositions do not provide the high volume of cold pressed organic safflower oil, or flaxseed oil, in their formulas to restore essential oil lipids that are essential to nourish and maintain healthy skin.

The said cited prior art compositions do not provide the high volume of skin healing vitamin A and tocopheral linoleate, to their compositions due to the prohibitively expensive manufacturing costs.

The said cited prior art compositions do not provide tincture of benzoin in their compositions.

The said cited prior art compositions do not boil tincture of benzoin to eliminate its alcohol content.

The said cited prior art compositions do not provide an urgent need that exists for compositions like the present invention to the dermatological, pharmaceutical, alternative therapy and cosmetology fields.

The said cited prior art compositions do not provide organic compositions to alleviate and treat the symptoms of lupus, and fungal skin infirmities.

The said cited prior art compositions do not provide organic compositions to cool and calm the skin.

The said cited prior art compositions do not provide organic non-irritating compositions to alleviate and treat persistent fungal foot infections.

The said cited prior art compositions do not provide organic antiseptic massage compositions to alleviate and treat dry skin conditions of the feet such as dermatitis, and eczema.

The said cited prior art compositions do not provide deep moisturizing foot care compositions while gently treating the skin for fungal infections.

The said cited prior art compositions do not provide organic moisturizing compositions that provide a water resistant, non-greasy, antiseptic film on the skin.

The said cited prior art compositions do not provide organic antibacterial compositions to prevent, treat and alleviate fungal foot infections.

The said cited prior art compositions do not provide organic compositions that are water resistant to body fluids, sebaceous gland activity and perspiration.

The said cited prior art compositions do not provide organic compositions to provide a high volume of organic essential oils for anti-inflammatory qualities and to perfume the said compositions.

The said cited prior art compositions do not provide a high volume of organic safflower and flaxseed oils in the said compositions.

The said cited prior art compositions do not provide high volume organic compositions to replace lost oil lipids to the foot dermis.

The said cited prior art compositions do not provide organic, gentle, moisturizing, non-irritating compositions to treat and alleviate persistent fungal foot conditions.

The said cited prior art compositions do not provide gentle, organic compositions for infant and adolescent use.

Objectives and advantages of the present invention are:

a) to provide an alcohol free topical skin preparation,

b) to provide essential oil as an organic antiseptic adjuvant to treat bacterial, fungal and environmental aggressors of the skin;

c) to provide topical foot composition that has potent effectiveness in the treatment of skin infirmities, such as dermatitis, eczema, lupus, chapping, rough skin, dryness, itching, cracking, flaking, and skin allergies related to synthetic substances and the like;

d) to provide a high volume of organic cold pressed vegetable oil composition for immediate transdermal delivery,

e) to provide a satisfactory skin penetration of the said composition and supplying properties that leave the skin satiny smooth for up to twelve hours,

f) to provide a high volume of organic essential oil for anti-inflammatory qualities and to perfume the said composition,

g) to provide a high volume of organic safflower and flaxseed oils in the said composition to replace oil lipids to the dermis,

h) to provide an urgent need that exists for composition like the present invention to the dermatological, pharmaceutical, alternative therapy and cosmetology fields,

i) to alleviate and treat the symptoms of atopic dermatitis, rough skin, dryness, cracking, atopic eczema, chapping, itching and allergic skin reactions due to ammonia, solvents, acids, alcohol substances, and synthetic perfumes and colorants;

j) to provide an organic beeswax substance that bonds lubricating, hydrating, moisturizing and antiseptic compounds to the skin for up to twelve hours;

k) to provide an organic calming, cooling, moisturizing composition to the feet,

l) to provide organic, gentle, moisturizing, non-irritating composition to treat and alleviate persistent fungal skin conditions,

m) to provide organic antibacterial composition to alleviate, treat, and prevent athletes foot infection,

n) to provide gentle, organic composition for infant and adolescent use,

o) to provide an organic water resistant, non-greasy, antiseptic and moisturizing film on the skin,

p) to provide an organic composition that is water resistant to body fluids, sebaceous gland activity and perspiration,

q) to provide an organic composition without chemical structures that are known to cause skin irritations; and

r) to provide organic compositions without solvents, perfumes, artificial colorants and ammonia additives.

Thus, an urgent need exists for skin composition like the present invention, that employs a high volume of potent cold-pressed organic flaxseed and safflower oils plus organic vitamins to feed the skin with nutritive polyunsaturated oil lipids without the addition of skin irritating synthetic adjuvants to achieve its purposes. Further advantages of this invention are the employment of an organic wax substance and an organic antiseptic substance that bonds to the skin to protect against fungal infections and treat environmental skin aggressors, leaving the skin moisturized and in a healthy condition for up to twelve hours. Still further objects and advantages will become apparent from a consideration of the ensuing descriptions.

### BRIEF SUMMARY OF THE INVENTION

This invention comprises an improved and useful topical transdermal delivery system for the alleviation and treatment of persistent foot infirmities, including dermatitis, eczema, lupus, dry, cracked, flaking, peeling, itching, bleeding, chaffed, and fungal skin conditions, which has as its constituents, safflower oil, flaxseed oil, vitamin A, tocopheryl linoleate, stearic acid, sweet almond oil, apricot kernel oil, cocoa butter, organic beeswax, jojoba oil, glycerin, essential oil of peppermint, liquid menthol, borax, and tincture of benzoin, in a cooling, hydrating, water-resistant, non-greasy composition.

### DETAILED DESCRIPTION OF THE INVENTION

It is an objective of the improved invention to provide potent, swift acting, non-sticky, organic, transdermal delivery formulation for treating and alleviating dry, distressed skin conditions, including dermatitis and eczema, lupus, rough skin, cracking, itching, peeling, redness, allergic skin reactions to alcohol and solvent sensitivity.

It is an objective of the improved invention to provide a skin formulation which soothes, moisturizers and restores essential nutrients to the skin to improve its texture and protect it against dryness.

It is an objective of the improved invention to provide an organic formulation for the treatment and alleviation of itching and redness, due to fungal infections of the feet.

It is an objective of the improved invention to provide an organic formulation for moisturizing and treating sensitive adolescent skin.

It is an objective of the improved invention to provide a high quantity of water in the formulation for the immediate transdermal delivery to the skin.

It is still a further objective of the improved invention to provide an organic formulation for the treatment, alleviation and maintenance of cracked, callused, rough, skin of the feet.

These and other objects are accomplished in accordance with a particular embodiment of the present invention which provides a foot care composition in the form of a cream, comprising a mixture consisting of tincture of benzoin, vitamin A and tocopheral linoleate, boric acid, organic beeswax, cold-pressed flaxseed, safflower oils, glycerin, borax, menthol, stearic acid, and essential plant oils and mixtures thereof. The essential components of the present composition readily intermix to form a mixture suitable for topical applications to the skin of the feet. The present components are combined in such relative proportions so as to bring about treatment for fungal infections, and a marked beautifying effect to the feet by supplying to the dermis nutrients that are essential for healthy skin.

The present improved invention includes the surprising discovery that cold-pressed organic safflower and flaxseed oils, tincture of benzoin, high volumes of vitamin A, and tocopheral linoleate, can be used as active principals in improved foot care composition to be employed to treat conditions attributed to lupus, eczema, dermatitis, and fungal foot conditions. In accordance with the present improved invention there is provided a number of formulations where cold-pressed, organic safflower oil, organic cold-pressed flaxseed oil, and tincture of benzoin can be employed with organic beeswax to aid in the water-resistant, non-greasy treatment and alleviation of chronic dry skin and fungal conditions. An embodiment of the improved invention follow to illustrate the formulations.

These main ingredients of my foot cream are:

50 to 800 milliliters water,

5 to 100 grams stearic acid,

10 to 100 grams cetearyl alcohol & polysorbate 60 called LipowaxP, purchased from Lipo Chemicals Inc, 207 19th Avenue, Paterson, N.J. 07504;

1 to 80 milliliters sweet almond oil,

0.1 to 100 milliliters cold-pressed safflower oil,

2 to 100 milliliters apricot kernel oil,

2.5 to 70 grams cocoa butter,

5 to 800 grams organic white beeswax,

1 to 100 milliliters jojoba oil,

2 to 100 milliliters glycerin,

00.1 to 100 milliliters cold-pressed flaxseed oil,

0.01 to 80 milliliters tocopheral linoleate,

0.1 to 50 milliliters composition containing 30,000 micrograms vitamin A/ml,

2 to 100 milliliters of a perfuming compound chosen from a group comprising oil of peppermint, spearmint, lemon, lime, orange, citrus, rose, cedar, rosemary, lavender, borage, sage, thyme, bergamot, cedar, and peach,

0.1 to 100 milliliters liquid menthol,

1 to 35 grams borax granules,

0.01 to 100 milliliters tincture of benzoin, and

0.1 to 10 milliliters dmdm hydantoin,

My foot cream process is as follows:

adding in a container being heated while said container is partially submersed in a 100 degree C. bath, safflower oil, sweet almond oil, apricot kernel oil, tocopheral linoleate, vitamin A, jojoba oil, flaxseed oil, cetearyl alcohol and polysorbate 60, beeswax, stearic acid, cocoa butter, and glycerin, to form a mixture;

stirring said mixture over hot water until dissolved,

adding simultaneously in a second container while said container is being heated while partially submersed in a 100 degree C. bath, water, borax, and tincture of benzoin;

stirring said mixture over hot water to allow the alcohol in the tincture of benzoin to burn off,

stirring said mixture over hot water until dissolved,

removing said mixtures from heat and blending both said mixtures until said mixture cools to 40 degrees C.;

adding perfuming compound, such as oil peppermint, liquid menthol, and a preservative such as dmdm hydantoin to said mixture;

blending said mixture until said mixture thickens, and whereby the ingredients are formed into a cream.

Whilst it is believed that the antiseptic, protective, softening, long term lubricating properties and the high volume of organic cold-pressed flaxseed oil lipids, and organic cold-pressed safflower oil lipids, and the high volume of vitamins A and tocopheral linoleate, borax, and tincture of benzoin, emulsified in organic beeswax are responsible for the alleviation, and treatment of dry skin infirmities, we don't wish to be bound by this.

As noted, the improved composition hereof comprise water-resistant, topical application that is applied to the dermal areas of the feet to alleviate and treat fungal infections, dry, chapped, flaky, rough, reddened, cracked, and chronic dry skin conditions, by feeding skin the essential polyunsaturated oil lipids it needs to maintain its healthy condition.

The improved formulation employ s organic polyunsaturated oil lipids in the place of mineral oil, which by its solvent action strips skin surface lipids.

This improved formulation treats skin in a moist and invisible manner.

For a more complete understanding of the present improved composition reference is made to the following examples. The following examples are illustrative of the present improved composition and are not intended in any manner as a limitation upon the scope thereof.

### EXAMPLE 1

The composition of the cream was applied twice daily to the feet of a woman suffering from dermatitis, which results in chronic, painful, dry skin. Within two days of use, the skin became soft and pliable, and the softening effects lasted all day.

### EXAMPLE 2

A male user found the foot composition to have enhanced absorption capabilities, and found it superior for swiftly alleviating his cracked dry feet condition.

### EXAMPLE 3

To evidence the efficacy of the foot composition, a series of individuals suffering from dry skin and cracked skin, were treated with the formulation. Compared to commercially available foot products, the results based upon the efficacy of the present invention are set forth hereinafter:

a) A female user found the present invention alleviated peeling skin cause by standing on her feet during her employment hours.

b) A female user found the present invention soothed and healed her itching, foot condition due to fungal infection.

c) A teenage female user found the present invention healed the deeply cracked skin on her feet, and kept the skin moist all day.

d) The present invention was used by a female who found that the skin on her feet did not crack during the summer months.

### EXAMPLE 4

A male user found that twice daily application of the foot cream, healed his deeply cracked heels, after which, a daily application kept his feet and heels, smooth, nourished and crack free.

### EXAMPLE 4

Male and female users reported that the foot soap cooled, soothed and alleviated their hot, painful, aching feet and legs, and the cooling effects lasted for up to an hour after each application. The present invention was used as an adjunct for treating foot fungus by the male and femnale users.

From the preceding, it will be readily perceived by the reader that an urgent need exists for these improved compositions for the alleviation of chronic skin conditions denoted heretofore, but which also, can be used to prevent such conditions from occurring;

The improved composition provides:

alcohol, silicone and solvent free formulations,

organic antiseptic adjuvant to fight bacterial, and fungal skin aggressors,

topical composition that has immediate effectiveness in the treatment of chronic skin infirmities,

superior skin penetration of the said composition and nutrient supplying properties,

organic anti-inflammatory qualities,

high volumes of organic polyunsaturated oils to replace lost dermal lipids,

fills an urgent need in the pharmaceutical, alternative health, and cosmetic markets,

organic beeswax to bond lubricating, hydrating, antiseptic and moisturizing compounds to the skin,

organic calming and cooling compounds,

deep moisturizing composition while treating fungal conditions,

composition that does not strip the skin of its natural oils,

a non-greasy moisturizing film on the skin that is resistant to attack by body fluids, sebaceous oils and perspiration,

light-weight formulation that is preferred for day time application,

organic oil lipids in the place of mineral oil, which by its solvent action strips skin surface lipids.

While the above description contains many specificity's, these should not be construed as limitations on the scope of the invention, but merely providing illustrations of some of the presently preferred embodiments of this invention.

Many other variations are possible. For example, it is additionally possible to add to the present invention many other adjuvants such as ultraviolet filters, color pigments, pearl pigments, additional essential oils and perfuming ingredients, vitamins, anibiotics, camphor, anti-inflammatory compositions, gelling agents, floral waters, and skin buffing agents and be within the purview of this invention.

Accordingly, the scope of the invention should be determined not by the embodiments illustrated, but by the appended claims and their legal equivalents, rather than by the examples given.

What is claimed is:

1. A cream comprising:
from 2 to 50 milliliters of sweet almond oil,
from 2 to 100 milliliters of safflower oil,
from 2 to 100 milliliters of apricot kernel oil,
from 2 to 120 milliliters of flaxseed oil,
from 0.01 to 50 milliliters of tincture of benzoin,
from 0.1 to 80 milliliters of tocopheral lineolate,
from 50 to 800 milliliters of water,
from 2 to 100 milliliters of jojoba oil,
from 10 to 100 grams cetearyl alcohol,
from 5 to 80 grams beeswax,
from 5 to 100 grams stearic acid,
from 2.5 to 70 grams cocoa butter,
from 0.5 to 50 milliliters composition containing 30,000 micrograms vitamin A/ml,
from 2 to 100 milliliters glycerin,
from 1 to 35 grams powdered borax,
from 0.1 to 10 milliliters of preservative such as dmdm hydantoin,
from 2 to 100 milliliters of a perfuming compound chosen from a group comprising, oil of peppermint, spearmint, lemon, lime, orange, citrus, rose, cedar, rosemary, lavender, borage, sage, thyme, bergamot, cedar, and peach,
from 0.1 to 100 liquid menthol.

2. A cream comprising:
135 milliliters water,
15 grams stearic acid,
45 grams cetearyl alcohol & polysorbate 60,
10 milliliters sweet almond oil,
10 milliliters cold-pressed safflower oil,
10 milliliters apricot kernel oil,
7.5 grams cocoa butter,
25 grams organic white beeswax,
5 milliliters jojoba oil,
5 milliliters glycerin,
10 milliliters cold-pressed flaxseed oil,
10 milliliters tocopheral linoleate,
10 milliliters composition containing 30,000 micrograms vitamin A/ml,
15 milliliters essential oil of peppermint,
15 milliliters liquid menthol,
1.25 grams borax granules,
5 milliliters tincture of benzoin, and
0.9 milliliters preservative such as dmdm hydantoin.

3. A method of making a cream comprising:
adding in a container being heated while said container is partially submersed in a 100 degree C. bath, safflower oil, sweet almond oil, apricot kernel oil, tocopheral linoleate, vitamin A, jojoba oil, flaxseed oil, cetearyl alcohol and polysorbate 60, beeswax, stearic acid, cocoa butter, and glycerin, to form a mixture;
stirring said mixture over hot water until dissolved, adding simultaneously in a second container while said second container is being heated while partially submersed in a 100 degree C. bath, water, borax, and tincture of benzoin;
stirring said mixture over hot water to allow the alcohol in the tincture of benzoin to burn off,
stirring said mixture over hot water until dissolved,
removing said mixtures from heat and blending both said mixtures until said mixtures cool to 40 degrees C.;
adding essential oil peppermint, liquid menthol, and dmdm hydantoin to said mixture;
blending said mixture until said mixture thickens, and
whereby the ingredients are formed into a cream.
